(19) United States
(12) Patent Application Publication
(10) Pub. No.: US 2013/0204154 A1
(43) Pub. Date: Aug. 8, 2013

(54) Signal Processing Device for use in Electroencephalography and a Cable System Incorporating the Device

(71) Applicants: Teck Loi, Chatswood (AU); Barry Clinch, Chatswood (AU); Bram Van Dun, Chatswood (AU)

(72) Inventors: Teck Loi, Chatswood (AU); Barry Clinch, Chatswood (AU); Bram Van Dun, Chatswood (AU)

(73) Assignee: HEAR IP PTY LTD, Melbourne, Victoria (AU)

(21) Appl. No.: 13/822,271

(22) PCT Filed: Aug. 24, 2011
(86) PCT No.: PCT/AU2011/001086

§ 371 Date: Apr. 19, 2013

(30) Foreign Application Priority Data
Sep. 13, 2010 (AU) 2010904096

(51) Int. Cl. A61B 5/04; A61B 5/0484; A61B 5/0478

(52) U.S. Cl. CPC A61B 5/04017; A61B 5/0478; A61B 5/04845
USPC 600/544

(57) ABSTRACT

A signal processing device for use in electroencephalography (EEG) is disclosed including: an input for receiving an electrical signal detected at a location on the head of a patient; at least one amplifier; a high-cut filter; and at least one output; wherein the signal is filtered by the high-cut filter prior to being amplified by the amplifier, the amplified signal being made available at the at least one output.

### TECHNICAL FIELD

The present invention relates to signal processing devices for use in electroencephalography and in particular to the initial amplification stage for such devices. The invention has particular application in assessing patients who have been fitted with cochlear implants.

### BACKGROUND TO THE INVENTION

External stimuli such as auditory events evoke electrical activity in the brainstem and auditory cortex. This activity can be detected by electroencephalography (EEG) whereby voltages on the scalp of the person hearing the sound are detected with electrodes placed on the scalp. EEG techniques have therefore found application in diagnosing patients with hearing problems. They find particular application in diagnosing infants and babies who do not have the language skills to explain to an audiologist what they are experiencing during a hearing test.

Such tests are used to diagnose the likely cause of hearing problems in a patient. In some cases, the diagnosis will point to either hearing aids or cochlear implants, or both, as a recommended treatment option. It is important that patients who may be assisted by cochlear implants receive these as early in life as possible, and preferably before they reach the age of 12 months. Furthermore, following being fitted with hearing aids or cochlear implants, a patient is usually subjected to hearing tests to monitor the effectiveness of the hearing aids or cochlear implants and make adjustments where necessary. It is possible to also use EEG techniques to perform this monitoring of effectiveness of the device.

Interference in EEG procedures is a long standing problem. Signals other than the signals of interest can be up to an order or larger in amplitude due to other brain activities and other sources are also picked up by the electrodes. The connecting wires between the scalp and the electronic circuit that detects the EEG voltages are susceptible to interference from power cables and other sources of electromagnetic noise in the environment, and this interference also contributes to the noise in the recording. Large common mode signals can be induced. Conventionally, a high-gain differential amplifier is employed to amplify signals picked up from two points on the scalp. This difference voltage is averaged synchronously with the repeated input sound to increase the signal-to-noise ratio of the evoked response relative to noise. Some devices incorporate amplification near the electrode sites in the form of high-gain amplifiers.

Recording subjects wearing a cochlear implant poses an additional problem as the signals transmitted through the skin to the implanted electronics, and the current sent by the implanted electronics to the electrodes situated in the cochlea can both cause large differential signals on the scalp that can overload the high-gain amplifiers. This generally results in a large artefact waveform that can obscure the evoked responses of the wearer. Alternatively, smaller artefact waveforms can have the same appearance as waveforms that normally emerge from the brain in response to sound, thus creating a false impression that the brain is reacting to the sound. This artefact is time locked to the acoustic stimuli and cannot be reduced by averaging. This artefact can be so large in amplitude as to make detection of the evoked response impossible.

There remains a need for improved an arrangement that can effectively detect cortical responses to auditory stimuli, particularly in subjects wearing cochlear implants.

### SUMMARY OF THE INVENTION

In a first aspect the present invention provides a signal processing device for use in electroencephalography (EEG) including: an input for receiving an electrical signal detected at a location on the head of a patient; at least one amplifier; a high-cut filter; and at least one output; wherein the signal is filtered by the high-cut filter prior to being amplified by the amplifier, the amplified signal being made available at the at least one output.

The high-cut filter may have a corner frequency of about 500 Hz.

The high-cut filter may have a corner frequency of about 200 Hz.

The high-cut filter may have a corner frequency of about 100 Hz.

The high-cut filter may have a corner frequency of about 50 Hz.

The device may be arranged to operate in an impedance measurement mode in which the input is connected to a resistance of known value which is in turn connected to an at least one output.

The signal processing device may be arranged to be put into the impedance measurement mode by way of a control signal issued by a control system.

The amplifier and low-pass filter may be packaged in a housing along with an EEG electrode connector, the input is in electrical connection with the electrode connector which is arranged to engage directly with an EEG electrode.

In a second aspect the present invention provides a cable system for use in electroencephalography (EEG), comprising a cable which is terminated at one end by a connector for connecting to an interface, and at the other end by a signal processing device according to the first aspect of the invention.

In a third aspect the present invention provides an arrangement of cable systems for use in electroencephalography (EEG) including first and second cable systems according to the second aspect of the invention, and wherein the signal processing devices are matched and their outputs combined to form a differential amplifier.

In a fourth aspect the present invention provides a method of measuring cortical brain activity of a patient including the steps of: detecting electrical signals at at least two locations on the head of a patient by way of at least two electrodes to produce a first active signal and a second reference signal; filtering the signals to attenuate the signals in a frequency range; amplifying and comparing the remaining portions of the active and reference signals to produce a measure of cortical brain activity.

The step of filtering may attenaute the signals above about 500 Hz.

The step of filtering may attenaute the signals above about 200 Hz.

The step of filtering may attenaute the signals above about 100 Hz.

The step of filtering may attenaute the signals above about 50 Hz.

The at least two electrodes may be connected to an interface unit by way of separate conductors.

### BRIEF DESCRIPTION OF THE DRAWINGS

An embodiment of the present invention will now be described, by way of example only, with reference to the accompanying drawings, in which:

FIG. 1 is a schematic representation of a first cable system for use in EEG according to the invention;

FIG. 2 is a schematic representation of a second cable system for use in EEG along with the cable system of FIG. 1; and

FIG. 3 is a photograph of one end of a cable system according to FIG. 1.

### DETAILED DESCRIPTION OF THE PREFERRED EMBODIMENT

Embodiments of the invention comprise a set of electrode cable systems comprising one or more active electrode cable systems, a reference electrode cable system and a ground electrode cable system. Each cable system has a terminator at both ends. The cable joining the end terminators is flexible, light weight, strengthened by felt or other material with similar physical properties, and contains a number of conductors including a shield. One terminator, the electrode terminator, incorporates miniature electronic components and is custom moulded with a snap connector that mates with the snap connector of an electrode attached to the scalp. The terminator at the other end connects to an interface unit that contains other follow on electronic circuits which may provide additional amplification, analogue to digital conversion, control, safety isolation and PC interface.

The electronic circuits in the reference electrode terminator can operate in one of two modes selectable by the interface unit with a control signal. In the impedance mode, the circuit returns a voltage that carries information on the impedance between the ground and reference electrode. This signal can be used to calculate the impedance between the ground and reference electrodes. In the response measurement mode, the circuit provides a low-impedance output of the signal picked up at the reference site. This signal with low output impedance is fed via the interface box to the active electrode terminator circuit to implement a high amplification of the difference between the reference and active signals.

Similarly, the electronic circuits in the active electrode terminator can operate in one of two modes selectable by the interface unit with a control signal. In the impedance mode, the circuit returns a voltage that carries information on the impedance between the ground and active electrode. This signal can be used to calculate the impedance between the ground and active electrodes. In the response measurement mode, the signal from the active electrode and the reference signal from the reference electrode terminator circuit are fed into a high-gain differential amplifier. A low-impedance output is fed down the cable to the interface unit.

The electronic circuit in the ground electrode terminator is limited to an ESD suppressor component. In impedance mode, an alternating voltage signal can be presented to the ground electrode by the interface unit. In response measurement mode, the ground electrode can be the conventional driven-right-leg electrode.

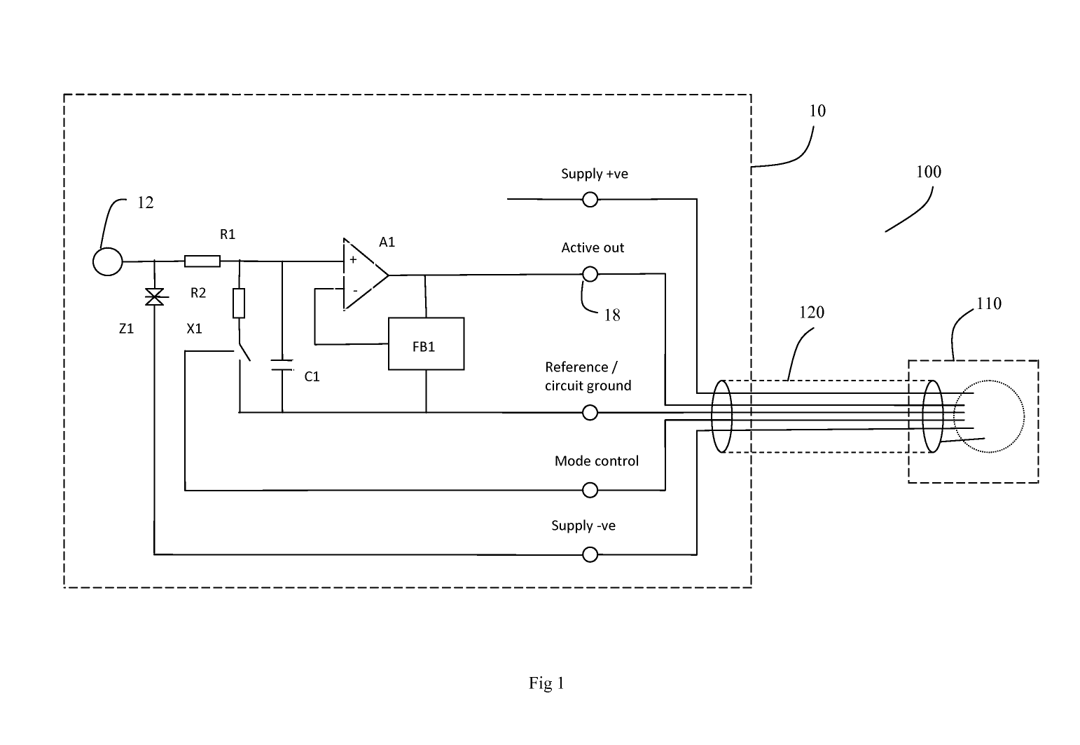

Referring to FIG. 1, an EEG cable system 100 is terminated at one end by a signal processing device 10, the device includes an EEG electrode input connector 12 to receive an electrical signal detected at a location on the head of a patient. Device further includes at least one amplifier in the form of operational amplifier A1, a high-cut filter in the form of capacitor C1, and an output 18.

Cable system 100 further includes a length of shielded cable 120 which is comprised of five conductors carrying power, mode control, reference/circuit ground and the active output signals as identified in the diagram. The other end of the cable is terminated by a seven pin Mini-DIN connector 110. The shield is connected to one of the pins, not to the jacket. Cable 120 is typically of about 1.5 metre in length. It is shown much shorter in the figure for ease of illustration.

The circuit can operate either in impedance measurement mode or response measurement mode as determined by the logic state of the mode control signal. In response measurement mode, switch X1 is open as shown. Amplification is set by the feedback network FB1 that acts as a voltage divider between A1's output and the reference signal originating from the reference electrode via a conductor in the electrode cables. FB1 in the preferred embodiment is set to provide an amplification of 121 times in the pass band of the signal picked up by the active electrode relative to the reference signal. Frequency shaping may be added in this feedback circuit. Z1 is an electrostatic discharge device included to protect the circuit by clamping the maximum voltage presentable to A1. R1 completes the protection by limiting the current that can flow into A1.

In impedance mode, the circuit in the interface unit switches circuit ground instead of reference to the terminator circuit. It also changes the mode control state to close switch X1. The interface circuit also applies a known alternating current signal on to the ground electrode. This voltage causes a current to flow through the scalp between the active and ground electrode and through R1 and R2. The latter is connected to circuit ground. In this mode the active out voltage can be used to calculate the resistance between the two electrodes as the forcing voltage at the ground electrode, R1 and R2 are known.

For cortical recordings when the subject is wearing a cochlear implant, artefacts caused by the cochlear implant are rejected by a high-cut filter, implemented with C1 placed right at the electrode pick up site. It is placed before the active circuit to eliminate overloading of A1. This significantly increases application of evoked response recordings (particularly cortical response recordings) in harsh conditions such as in close proximity to cochlear implants. In the embodiment preferred for application in cortical response testing, the corner frequency as set by the source impedance in series with R1 and C1 varies advantageously with electrode contact condition. It is lower with poorer connections (higher impedance) thereby rejecting more noise and artefacts. With good electrode connections, source impedance of around 5 kΩ, the corner frequency is set at 50 Hz. In the circuit shown in FIG. 1, suitable values for the components are as follows: R1=10.0Ω, R2=270Ω, C1=0.22 μF.

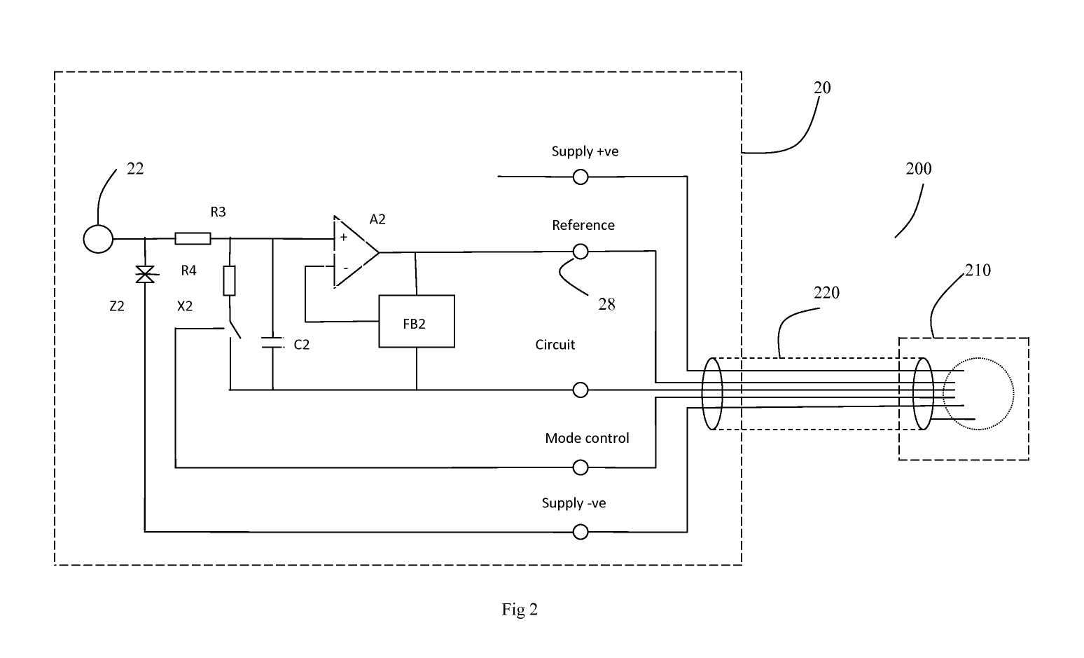

Referring to FIG. 2, the cable system used for the reference electrode is illustrated and is similar in many respects to the active electrode cable system. Again, the shielded cable 220 is terminated at both ends. At one end is a signal processing device 20, at the other, a seven pin Mini-DIN plug 210. The shielded cable includes five conductors carrying power, mode control circuit ground and reference output signal as identified in the diagram. A2 is an operational amplifier.

The circuit can operate either in impedance measurement mode or response measurement mode as determined by the logic state of the mode control signal. In response acquisition mode, switch X2 is open as shown. Amplification is set by the feedback network FB2, a voltage divider between A2′s output and the circuit ground. FB2 in the preferred embodiments is set to provide an amplification of just over unity (121 divided by 120). It is matched to the amplification of the active electrode terminator circuit to achieve a high common mode rejection and a high differential amplification. Z2 is an electrostatic discharge device included to protect the circuit by clamping the maximum voltage presentable to A2. R3 completes the protection by limiting the current that can flow into A2.

In impedance mode, the interface unit changes the mode control state to close switch X2. The interface circuit also applies a known alternating current signal to the ground electrode. Similar in operation to the active terminator circuit, in this mode the reference output voltage can be used to calculate the resistance between the ground and reference electrodes as the forcing voltage at the ground electrode, R3 and R4 are known.

For cortical recordings when the subject is wearing a cochlear implant, artefacts due the cochlear implant are rejected by a high-cut filter, implemented with C2 placed right at the electrode pick-up site. It is placed before the active circuit to eliminate saturation of A2. This significantly increases application of evoked response recordings (particularly in cortical response recordings) in harsh conditions such as in close proximity to cochlear implants. In the embodiment preferred for application in cortical response testing, the corner frequency as set by the source impedance and R3 in series with C2 varies advantageously with electrode contact condition. It is lower with poorer connections (higher impedance) thereby rejecting more noise and artefacts. With good electrode connections, source impedance of around 5 kΩ, the corner frequency is set at 50 Hz.

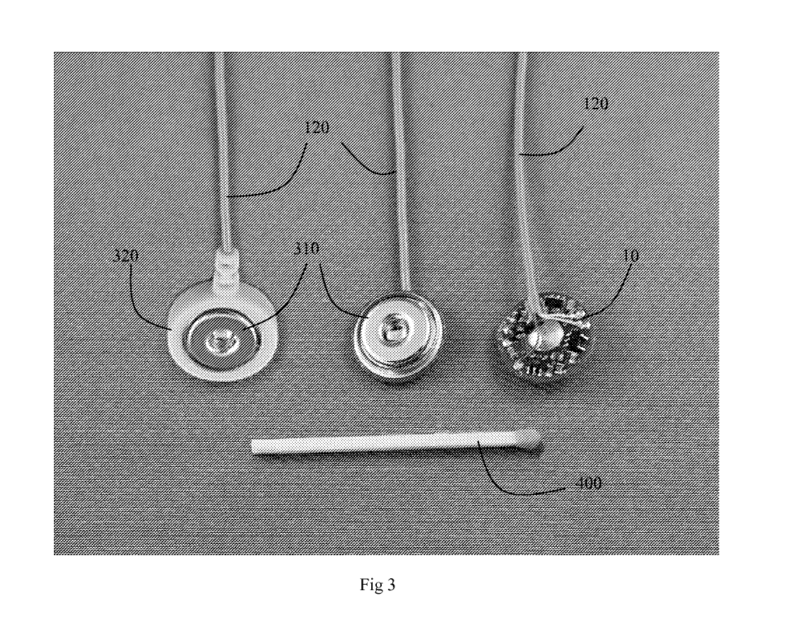

Referring to FIG. 3, the internal construction of a preferred embodiment of an electrode terminator 10 is shown alongside a matchstick, 400, shown to give an idea of scale. Surface mount miniature components are mounted on one side of a thin printed circuit substrate and the EEG electrode connector 310 is mounted on the opposite side of the substrate and directly connected to circuit input 12 (see FIG. 1). The finished termination is covered by a moulded plastic housing 320. The connector 310 remains exposed and is a snap-fit with a disposable self-adhesive EEG electrode. This arrangement eliminates any wiring between the electrode and the electronic circuit.

It can be seen that embodiments of the invention have at least one of the following advantages:
Can be used to monitor cortical responses to auditory stimuli in patients wearing cochlear implants
The signal processing devices can operate in an impedance mode to ensure that adequate electrode contact is achieved before testing
Locating the processing device at the site of the electrode reduces interference

It will be evident to those skilled in the art that the separate cable systems can be combined into a single cable system in which the separate conductors are incorporated within a single cable terminated by an interface terminator, or wireless transmitter at one end, and by separate electrode connectors at the other end.

Any reference to prior art contained herein is not to be taken as an admission that the information is common general knowledge, unless otherwise indicated.

Finally, it is to be appreciated that various alterations or additions may be made to the parts previously described without departing from the spirit or ambit of the present invention.

1-16. (canceled)

17. A signal processing device for use in electroencephalography (EEG) including:
an input for receiving an electrical signal detected at a location on the head of a patient;
at least one amplifier;
a high-cut filter; and
at least one output;
wherein the signal is filtered by the high-cut filter prior to being amplified by the amplifier, the amplified signal being made available at the at least one output; and
wherein the amplifier and high-cut filter are packaged in a housing along with an EEG electrode connector, the input is in electrical connection with the electrode connector which is arranged to engage directly with an EEG electrode.

18. A signal processing device according to claim 17 wherein the high-cut filter has a corner frequency of about 500 Hz.

19. A signal processing device according to claim 17 wherein the high-cut filter has a corner frequency of about 200 Hz.

20. A signal processing device according to claim 17 wherein the high-cut filter has a corner frequency of about 100 Hz.

21. A signal processing device according to claim 17 wherein the high-cut filter has a corner frequency of about 50 Hz.

22. A signal processing device according to claim 17 which is arranged to operate in an impedance measurement mode in which the input is connected to a resistance of known value which is in turn connected to an at least one output.

23. A signal processing device according to claim 22 which is arranged to be put into the impedance measurement mode by way of a control signal issued by a control system.

24. A cable system for use in electroencephalography (EEG), comprising a cable which is terminated at one end by a connector for connecting to an interface, and at the other end by a signal processing device according to claim 17.

25. An arrangement of cable systems for use in electroencephalography (EEG) including first and second cable systems according to claim 24, and wherein the signal processing devices are matched and their outputs combined to form a differential amplifier.

26. A method of measuring cortical brain activity of a patient including the steps of:
detecting electrical signals at at least two locations on the head of a patient by way of at least two electrodes directly engaged to the electrode connectors of respective signal processing devices of an arrangement of cable systems according to claim 9 to produce a first active signal and a second reference signal;
filtering the signals to attenuate the signals in a high frequency range;
amplifying and comparing the remaining portions of the active and reference signals to produce a measure of cortical brain activity.

27. A method according to claim 26 wherein the step of filtering attenuates the signals above about 500 Hz.

28. A method according to claim 26 wherein the step of filtering attenuates the signals above about 200 Hz.

29. A method according to claim 26 wherein the step of filtering attenuates the signals above about 100 Hz.

30. A method according to claim 26 wherein the step of filtering attenuates the signals above about 50 Hz.
